(19) United States
(12) Patent Application Publication
(10) Pub. No.: US 2011/0086426 A1
(43) Pub. Date: Apr. 14, 2011

(54) METHODS AND APPARATUS FOR COLLECTING AND SEPARATING REGENERATIVE CELLS FROM ADIPOSE TISSUE

(75) Inventor: Robert Michael Freund, Old Westbury, NY (US)

(73) Assignee: Lipostem Corp., Greenwich, CT (US)

(21) Appl. No.: 12/578,006

(22) Filed: Oct. 13, 2009

(51) Int. Cl. C12N 5/071; C12M 3/08

(52) U.S. Cl. 435/379; 435/308.1

(57) ABSTRACT

Methods and apparatus for: collecting adipose tissue in a syringe; subjecting the collected adipose tissue to heat, vibration, and or centrifugation whilst remaining within the syringe; and filtering the adipose tissue during centrifugation such that the regenerative cells are permitted to pass into a reservoir of a collection sleeve.

### BACKGROUND OF THE INVENTION

The present invention relates to methods and apparatus for separating and concentrating regenerative cells (e.g., stem cells), from adipose tissue.

Regenerative cells, e.g., stem cells and/or progenitor cells (i.e., the unspecialized master cells of the body), renew themselves indefinitely and develop into mature specialized cells. Stem cells are found in embryos during early stages of development, in fetal tissue and in some adult organs and tissue. Embryonic stem cells (ESCs) are known to become many, if not all, of the cell and tissue types of the body. ESCs not only contain all the genetic information of the individual in which they are produced but also contain the nascent capacity to become any of the 200+cells and tissues of the body. Thus, ESCs have potential for regenerative medicine. For example, ESCs can be grown into specific tissues for particular body organs, such as the heart, lungs or kidneys, which may be used to repair damaged and diseased organs. However, tissues derived from ESCs have clinical limitations. Since ESCs are necessarily derived from another individual, i.e., an embryo, there is a risk that the recipient's immune system will reject the new biological material. Although immunosuppressive drugs are available to prevent such rejection, such drugs are also known to block desirable immune responses, such as those against bacterial infections and viruses. Moreover, the ethical debate over damage done to the life from which the ESCs are taken, i.e., the embryo, is well-chronicled and presents an insurmountable moral obstacle.

Adult stem cells (ASCs) represent a viable alternative to the use of ESCs. ASCs reside quietly in many non-embryonic tissues, presumably waiting to respond to trauma or other destructive disease processes so that they can heal the injured tissue. Notably, emerging scientific evidence indicates that each individual carries a pool of ASCs that, like ESCs, have the ability to become many, if not all, types of cells and tissues of the individual in which they are produced. Thus, ASCs, like ESCs, have tremendous potential for clinical applications of regenerative medicine.

Sources of ASCs include bone marrow, skin, muscle, liver and brain tissues. However, the concentration of ASCs in these tissues is considered relatively low. For example, mesenchymal stem cell concentration in bone marrow is estimated at between 1 in 100,000 and 1 in 1,000,000 nucleated cells. Similarly, extraction of ASCs from certain of these tissues is difficult. For example, extracting ASCs from skin involves a complicated series of cell culture steps over several weeks, and clinical application of skeletal muscle-derived ASCs requires a two to three week culture phase. Thus, any proposed clinical application of ASCs from such tissues requires increasing cell number, purity, and maturity by processes of cell purification and cell culture.

Although cell culture steps may increase the number of available ASCs, the purity, and the maturity, they do so at a significant cost. The cost and associated problems may include one or more of the following: loss of cell function due to cell aging, loss of potentially useful non-stem cell populations, delays in potential application of cells to patients, increased monetary cost, and increased risk of contamination of cells with environmental microorganisms during culture. Recent studies examining the therapeutic effects of bone-marrow derived ASCs have used essentially whole marrow to circumvent the problems associated with cell culturing. The clinical benefits, however, have been suboptimal, an outcome believed related to the limited ASC concentration and purity inherently available in bone marrow.

Adipose tissue has also been shown to be a source of ASCs. Unlike marrow, skin, muscle, liver and brain tissues, adipose tissue is comparably easy to harvest in relatively large amounts. Furthermore, adipose derived ASCs have been shown to possess the ability to generate multiple tissues in vitro, including bone, fat, cartilage, and muscle. Thus, adipose tissue presents an optimal source for ASCs for use in regenerative medicine.

Suitable methods for harvesting adipose derived ASCs, however, have been lacking in the art. Indeed, existing methods may suffer from a number of shortcomings, including an inability to optimally accommodate an aspiration device for removal of adipose tissue, a lack of partial or full automation from the harvesting of adipose tissue phase through the processing of tissue phases, a lack of a partially or completely closed system from the harvesting of adipose tissue phase through the processing of tissue phases, significant risks of cross-contamination of material from one sample to another, high processing costs (including complex and expensive equipment), and long cycle times from harvest to cell availability for clinical use.

Accordingly, there remains a need in the art for systems and methods that are capable of harvesting regenerative cell populations, e.g., ASCs, with increased yield, consistency and/or purity, and of doing so rapidly and at low cost. A related need is for the system and method to yield regenerative cells in a manner suitable for direct placement into a recipient.

### SUMMARY OF THE INVENTION

In accordance with one or more embodiments of the present invention, methods and apparatus provide for: collecting adipose tissue in a syringe, the syringe including a body having an internal chamber, a proximal end through which a plunger assembly slides into and out of the chamber, and a distal end through which the adipose tissue is drawn into the chamber; inserting the body of the syringe into an open, proximal end of a collection sleeve such that the distal end of the syringe is in fluid communication with a reservoir at an opposing, closed end of the collection sleeve; subjecting the collected adipose tissue to heat and vibration whilst remaining within the syringe in the collection sleeve to initiate separation of the adipose tissue into strata, where a concentration of the regenerative cells are in a first of the strata and a substantial concentration of fat is in a second of the strata; subjecting the syringe and collection sleeve to centrifugation such that regenerative cells and some secondary materials in the first stratum are drawn toward and out of the distal end of the chamber of the syringe; and filtering the first stratum such that the regenerative cells are permitted to pass to the reservoir of the collection sleeve in response to the centrifugation.

The methods and apparatus may further provide for adding a cell separation enzyme to the collected adipose tissue within the syringe prior to heat and vibration.

The methods and apparatus may further provide for coupling the distal end of the syringe to a mating end of a filter disposed within the collection sleeve, the filter closing off the reservoir of the collection sleeve from the open, proximal end thereof, wherein the filter performs the filtering step by permitting the regenerative cells to pass through the mating end, through an output end thereof, and into the reservoir of the collection sleeve, but prohibits at least some of the secondary material from passing therethrough.

The methods and apparatus may further provide for: inserting the collection sleeve, the syringe, and the adipose tissue therein into a fluid chamber of a centrifuge; and elevating a temperature of fluid within the fluid chamber of the centrifuge to a predetermined temperature for a time sufficient to at least initiate separation of the adipose tissue into the strata. Preferably, the collection sleeve, the syringe, and the adipose tissue therein are subject to vibration whilst in the centrifuge. Preferably, the vibration results in orbital shaking of the adipose tissue. The predetermined temperature may be about 37° C. The time for heating and vibration may be about 30 minutes.

Other aspects, features, and advantages of the present invention will be apparent to one skilled in the art from the description herein taken in conjunction with the accompanying drawings.

### DESCRIPTION OF THE DRAWINGS

For the purposes of illustration, there are forms shown in the drawings that are presently preferred, it being understood, however, that the invention is not limited to the precise arrangements and instrumentalities shown.

FIG. 1 is a schematic diagram of a syringe for collecting adipose tissue (including ASCs), suitable for use in connection with one of more embodiments of the present invention;

FIG. 2 is a schematic diagram of the syringe of FIG. 1, where access through a plunger thereof is provided to insert an additive to the collected adipose tissue;

FIGS. 3A and 3B are schematic views of the plunger and plunger shaft of the syringe of FIG. 1 in assembled and unassembled configurations, respectively;

FIGS. 3C and 3D are rear and front views, respectively, of the plunger head of FIG. 3B;

FIGS. 4A and 4B are schematic diagrams of the syringe of FIG. 1 and a collection sleeve in unassembled and assembled configurations, respectively;

FIGS. 5A and 5B are diagrams of a filter element of the collection sleeve viewed from input and output sides, respectively;

FIG. 6 is a perspective view of a centrifuge suitable for use in connection with one or more aspects of the present invention;

FIG. 7 is a schematic diagram of the centrifuge of FIG. 6 showing additional details;

FIG. 8 is a schematic view of a vibration mechanism that operates to provide vibration energy to the centrifuge of FIGS. 6 and 7;

FIG. 9A is a schematic diagram of the syringe and collection sleeve in an assembled configuration after heat and/or vibration processing, which initiates separation within the syringe;

FIG. 9B is a schematic diagram of the syringe and collection sleeve in an assembled configuration after centrifugation, where ASCs are concentrated within a reservoir of the collection sleeve;

FIG. 10 is a schematic diagram of the collection sleeve (with the syringe removed) in a sealed configuration for temporary storage of the collected ASCs;

FIGS. 11A-11B show a top view, and a cross-sectional view, respectively, of an alternative filter, which includes features that permit opening and closing of the syringe;

FIGS. 12A-12B show the filter of FIG. 11 in engagement with the syringe in open and closed configurations, respectively;

FIG. 13 is a side view of a collection sleeve having an alternative configuration in accordance with one or more further aspects of the present invention;

FIG. 14 is a top view of a rotation mechanism having an alternative configuration, suited for receiving the collection sleeve of FIG. 13; and

FIG. 15 is a side sectional view of a universal ring suitable for use in connection with the collection sleeve of FIG. 13.

### DETAILED DESCRIPTION OF THE PREFERRED EMBODIMENTS

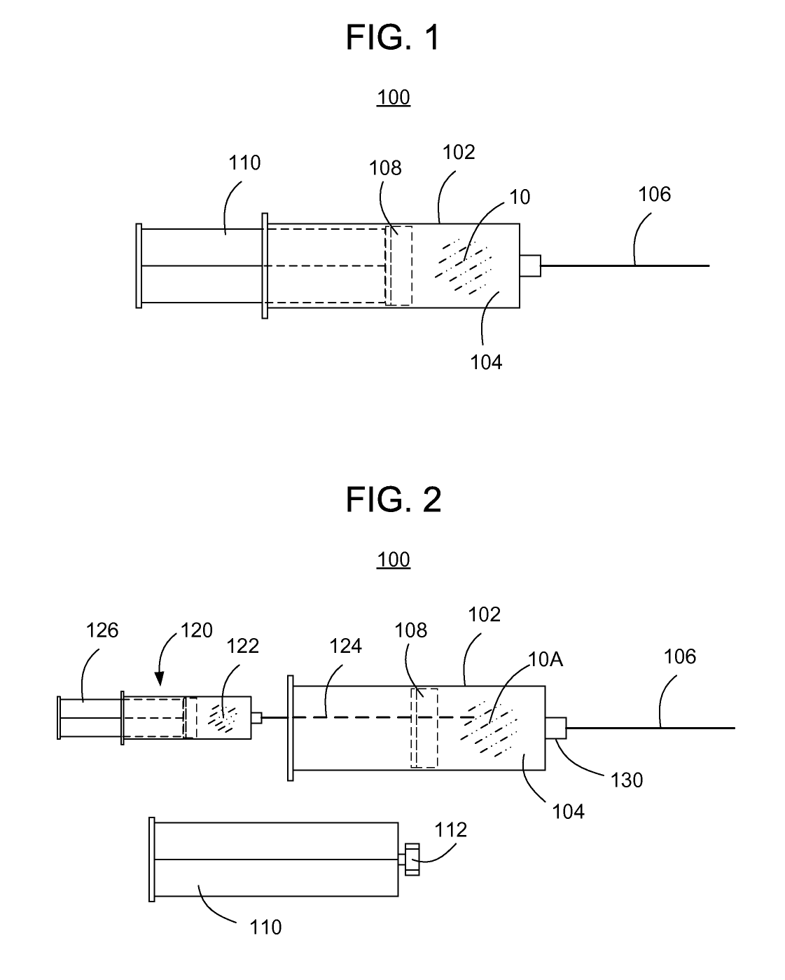

With reference to the drawings, wherein like numerals indicate like elements, there is shown in FIG. 1 a device for collecting adipose tissue (and the ASCs therein) from a living organism. In the illustrated embodiment, the device is a syringe 100, including a body 102 defining an internal chamber 104, a needle or cannula 106 in fluid communication with the internal chamber 104, and a plunger assembly 108, 110 in sliding engagement with the internal chamber 104. The body 102 includes a proximal end through which the plunger assembly 108, 110 slides into and out of the chamber 104, and a distal end (or luer end) to which the needle 106 is connected and through which the adipose tissue 10 is drawn into the chamber 104.

The plunger assembly includes a plunger 108 and a plunger shaft 110. The movement of the plunger 108 within the chamber 104 varies an interior volume thereof. For example, movement of the plunger 108 in the proximal direction increases the volume of the chamber 104 and generates a vacuum or suction at the needle 106 to harvest the adipose tissue 10 into the chamber 104. Movement of the plunger 108 in the distal direction decreases the interior volume of the chamber 104, and pushes material out of the chamber 104 and through the needle 106.

The collection of the adipose tissue 10 may include injecting a fat harvesting site in a patient with tumescent fluid (saline with adrenaline and lidocaine). The tumescent fluid stiffens the fat and reduces bleeding and discomfort. The needle 106 of the syringe 100 is then inserted into the fat harvesting site and the adipose tissue 10, including a combination of oil, fat (such as fat tissue or fat cells), tumescent fluid, ASCs, and other substances are drawn through the needle 106 into the chamber 104. The adipose tissue 10 may be harvested by aspiration, by pulling the plunger shaft 110 and plunger 108 in the proximal direction to draw the tissue 10 up through the needle 106 into the chamber 104 of the syringe 100.

Next, a cell separation enzyme may be added to the collected adipose tissue 10 within the syringe 100. Such cell separation enzymes are collagenases, which are enzymes that break the peptide bonds in collagen. A suitable collagenase is xiaflex from Biospecifics Technologies Corp., which is an FDA approved product containing collagenase as its primary ingredient. One approach is to draw the cell separation enzyme from a sterile container into the chamber 104 through the needle 106.

Another approach is to introduce the cell separation enzyme into the chamber 104 using another syringe 120. In particular, the other syringe 120 may be used to draw the cell separation enzyme 122 from a sterile container (not shown) into a chamber thereof. Next, a needle 124 of the syringe 120 is driven through the plunger 108 and into the chamber 104 of the syringe 100. Then the cell separation enzyme 122 is discharged into the chamber 104 by activating the plunger shaft 126 of the other syringe 120.

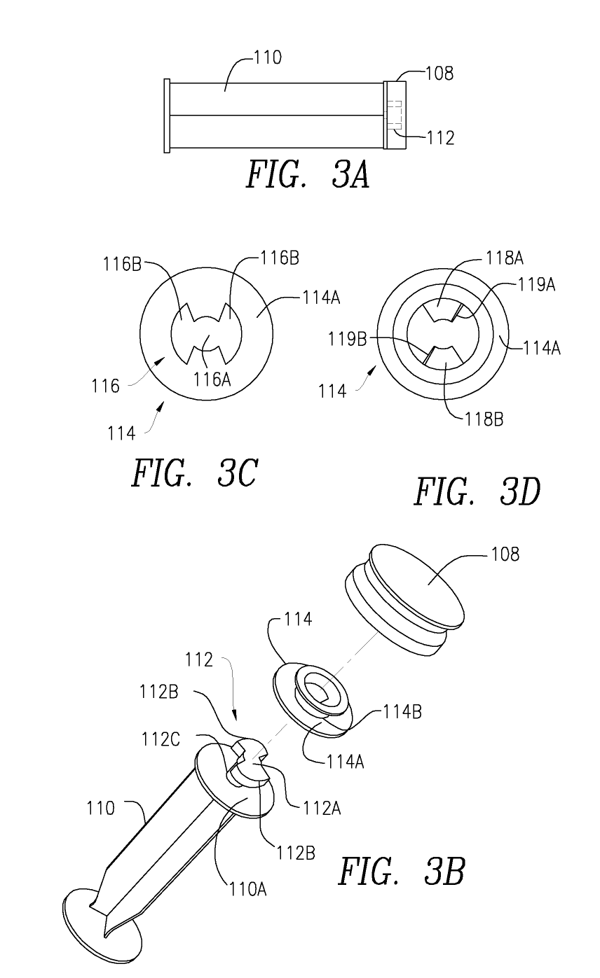

With reference to FIGS. 3A-3D, the syringe 100 may include a structure that exposes a passage for inserting the needle 124 of the syringe 120 through the plunger 108 and into the chamber 104 thereof. In particular, the plunger 108 may be releasably coupled to the plunger shaft 110. Such coupling is achieved by way of a coupling element 112 of the shaft 110 and a corresponding mating element of the plunger 108. More specifically, the plunger 108 may be coupled to a plunger head 114, which is generally formed of a relatively stiff material (such as a suitable plastic) as compared with the resilient material of the plunger 108. The plunger head 114 is of a size and shape to apply thrusting pressure and drawing forces to the plunger 108 within the cavity 104 of the syringe 100. The plunger head 114 includes a rear plate 114A, which is engaged by a drive plate 110A of the plunger shaft 110. A throat 114B extends from the rear plate 114A and terminates at an overhanging, annular lip 114C. A cavity within the plunger 108 is sized and shaped to receive the throat 114B and the lip 114C, with the lip 114C mating with an undercut of the plunger cavity to ensure that the plunger 108 does not easily disengage from the plunger head 114.

With specific reference to FIG. 3B, the coupling element 112 may extend from the drive plate 110A of the plunger shaft 110. Among the various suitable configurations, the coupling element 112 may be of a bow-tie shape, including a central portion 112A and oppositely extending wedge-shaped projections 112B. The central portion 112A and projections 112B may be offset from the drive plate 110A by way of a relatively short shaft 112C.

With specific reference to FIGS. 3C and 3D, the plunger head 114 includes an aperture 116 that is sized and shaped to receive the coupling element 112 of the plunger shaft 110. Thus, the aperture 116 has a corresponding bow-tie shape, including a central portion 116A and oppositely extending wedge-shaped receptacles 116B. To engage the plunger shaft 110 with the plunger head 114 (with the plunger 108 in place), the coupling element 112 is inserted through the aperture 116 from the rear side of the rear plate 114A, aligning the oppositely extending wedge-shaped projections 112B of the coupling 112 with the oppositely extending wedge-shaped receptacles 116B of the aperture 116. Once inserted, a twisting motion of the plunger shaft 110 relative to the plunger head 114 rotates and slides the wedge-shaped projections 112B within the plunger head 114 against bearing surfaces 118A, 118B until the projections 112B come to rest against stops 119A, 119B. In such position, the plunger head 114 and plunger 108 may be driven into and out of the chamber 104 of the syringe 100.

To disengage the plunger shaft 110 from the plunger head 114, the above steps are reversed. When the collected adipose tissue 10 is within the chamber 104, and the plunger shaft 110 is disengaged from the plunger head 114, a rear side of the resilient plunger 108 is exposed through the aperture 116 of the plunger head 114. Thus, the needle or cannula 124 of the other syringe 120 may be inserted through the open end of the body 102 of the syringe 100, through the aperture 116 of the plunger head 114, and through the resilient material of the plunger 108 into the chamber 104. Then the cell separation enzyme 122 may be injected into the collected adipose tissue 10.

Either of the above approaches results in a mixture of materials 10A (FIG. 2) within the chamber 104 of the syringe 100, including a combination of oil, fat, tumescent fluid, ASCs, cell separation enzyme, and other substances. As will be discussed below, it is desirable to separate at least some of these materials into strata so that the ASCs and other useful materials (such as the viable fat) may be collected for clinical purposes.

Prior to or after the introduction of the cell separation enzyme into the syringe 100, the needle or cannula 106 may be removed. By way of example, the needle 106 may include a threaded coupling at a proximal end thereof that connects and disconnects (e.g., via threads) with a corresponding coupling 130 of the syringe 100.

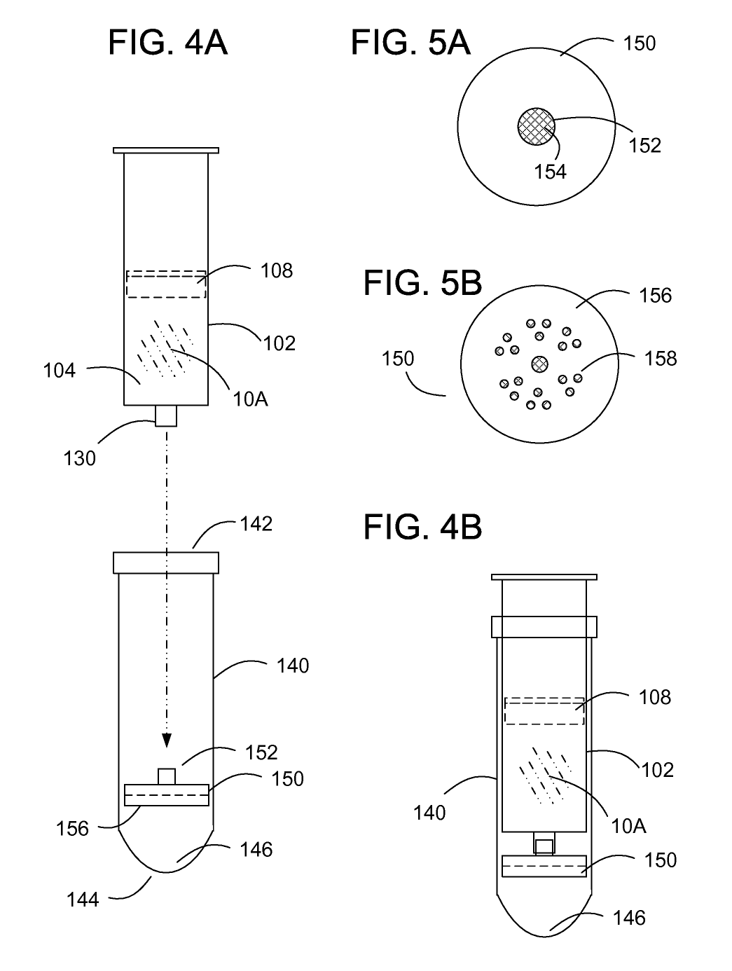

To assist in the processing of the material 10A, the system may include a collection sleeve 140. With reference to FIGS. 4A and 4B, the collection sleeve 140 may include an open proximal end 142, and a closed, distal end 144. A filter assembly 150 is disposed within the collection sleeve 140 and separates an interior volume thereof into an open, proximal end and a closed-off reservoir 146. With additional reference to FIGS. 5A and 5B, the filter 150 includes an input end and an output end, the input end including a coupling 152 and corresponding aperture in fluid communication with an interior volume. The output end 156 is disposed towards the reservoir 146 and includes one or more apertures 158 also in fluid communication with the interior volume of the filter 150. A mesh film 154 is disposed within the interior volume of the filter 150, separating the input end from the output end thereof. The mesh 154 operates to pass material having a range of particle sizes and to block other material having another range of particle sizes. By way of example, the mesh 154 may include a pore size of between about 50 um to about 150 um. In an alternative configuration, the mesh 154 may include a pore size of about 500 um.

The body 102 of the syringe 100 is inserted into the open end 142 of the collection sleeve 140 such that the coupling 130 at the distal end of the syringe 100 mates with the coupling 152 at the input end of the filter 150. By way of example, the coupling 130 of the syringe 100 and the coupling 152 of the sleeve 140 may include complementary threads (one male and one female) that permit the requisite connection and disconnection. Preferably, such connection is fluid tight. Thus, when coupled together, the distal end of the syringe 100 is in fluid communication with the reservoir 146 of the sleeve 140 via the input and output ends of the filter 150.

The mixture of materials 10A is preferably subject to heat and vibration whilst remaining within the syringe 100 to at least initiate separation of the material 10A into strata.

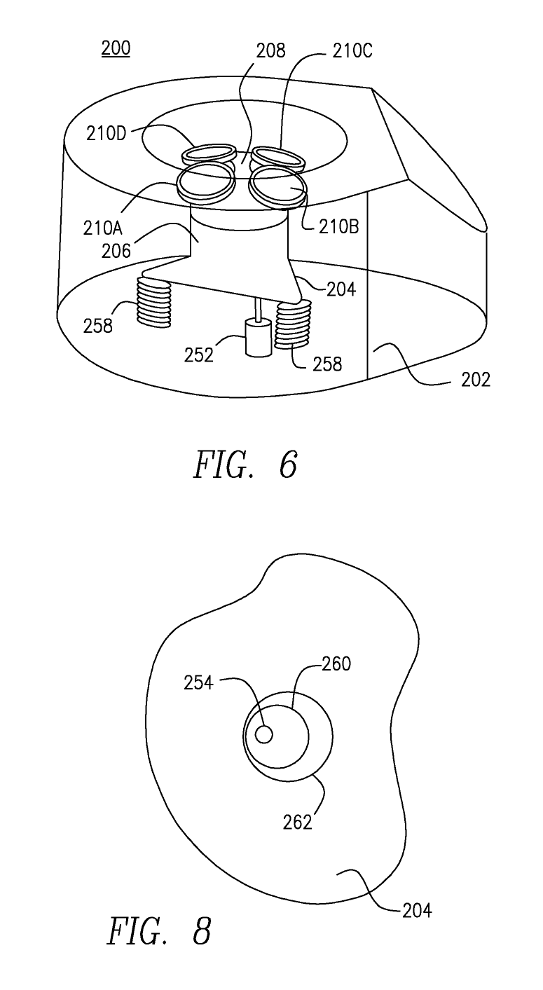

The aforementioned heat and/or vibration may be applied to the material 10A while the syringe 100 is within the collection sleeve 140. To that end, and with reference to FIGS. 6-8, the system may include a centrifuge 200 that also includes a heating and vibration capability. the centrifuge 200 may include a base 202, an intermediate platform 204 coupled to the base 202, a rotor housing 206 coupled to the intermediate platform 204, and a rotor mechanism 208 rotatable relative to the rotor housing 206. The centrifuge 200 is preferably electrically operated (e.g., via battery and/or other A/C or D/C source). In such an embodiment, the centrifuge includes a rotary motor 206A within the rotor housing 206, which is coupled to the rotor mechanism 208 via a shaft 206B. Thus, application of energy to the rotor motor 206A causes rotation of the shaft 206B and the rotor mechanism 208. In alternative embodiments, the centrifuge 200 may be manually driven, in which case the rotary motor 206A may be replaced with a hand-crank linkages, which are well known in the art.

Although application of energy to the rotary motor 206A may be achieved in many different ways, using existing circuitry, it is preferred that such energy is controlled in at least a partially automated fashion. In particular, it is preferred that a control system automatically energizes the rotary motor 206A in order to achieve a desired rotational speed (or speeds if a profile is desired) and a desired duration (or durations, again, if a profile is desired). To this end, the control system may include a microprocessor 220, a memory 222 and one or more interfaces 224. The memory 222 may contain programs and/or data needed to cause the micro-processor 222 to carry out certain actions, such as turning on the rotary motor 206A, causing same to rotate at a particular speed or speeds, turning off the rotary motor 206A, etc. The interfaces 224 contain suitable circuitry to receive digital control signals from the micro-processor 220, convert same to analog signals (if needed) and send such signals (e.g., signals on line 207) to the rotary motor 206A. Skilled artisans may readily obtain or produce suitable computer-readable programs and/or data, as well as the circuitry of the interfaces 224 to achieve such functionality.

In the event that user input is required, such as to set rotation speed(s), rotation profiles, duration(s), etc., an input device 226 (such as a keyboard or the like) is coupled to the micro-processor 220.

It is noted that the microprocessor 220, memory 222, interfaces 224, and or input device 226 may be implemented utilizing any of the known technologies, such as standard digital circuitry, analog circuitry, microprocessors, digital signal processors, any of the known processors that are operable to execute software and/or firmware programs, programmable digital devices or systems, programmable array logic devices, or any combination of the above, including devices now available and/or devices which are hereinafter developed.

The rotor mechanism 208 includes one or more couplings 210 for receiving one or more sleeves 140 to be subject to heating, vibration, and/or centrifugation. In the illustrated embodiment, there are four such couplings 210A, 210B, 210C, and 210D, although any number may be employed. Among the many suitable configurations, each of the couplings 210 includes a swiveling ring of a diameter suitable to receive a collection sleeve 140 therein, but not so large that a peripheral rim at the proximal end 142 of the sleeve 140 will pass therethrough. Thus, as the shaft 206B and the rotor mechanism 208 rotate, the rings will tend to swivel such that the distal ends of the sleeves 140 will travel through a larger and larger path, thereby ensuring that centrifugal forces drive the material 10A out of the distal end of the body 102 of the syringe 100, through the couplings 130, 152 and toward the mesh 154 of the filter 150. The fact that the couplings 102 are of a ring-type also facilitates the application of heat to the collection sleeve 140, thereby heating the material 10A within the syringe 100. This feature will be discussed in more detail below.

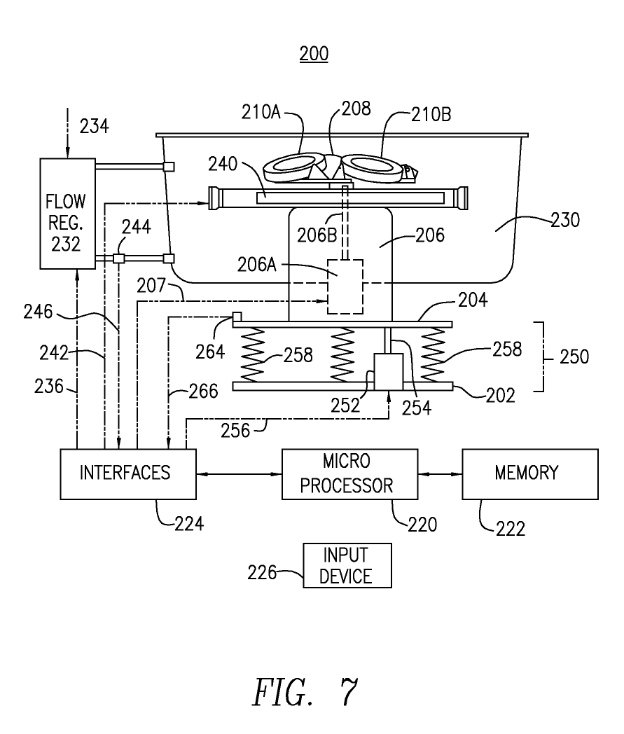

As best seen in FIG. 7, the centrifuge 200 may include a fluid chamber 230 surrounding the rotor mechanism 208. In the illustrated embodiment, the fluid chamber 230 is of a size suitable to encompass the rotor mechanism 208 and at least a portion of the rotor housing 206, although other sizes and shapes are well within the scope of the invention. The fluid chamber 230 is preferably sealed to the extent necessary to ensure that the particular type of fluid introduced into the chamber 230 does not leak out or escape at all (e.g., in the case of a liquid fluid) or at least to a desired degree (e.g., in the case of a gas fluid). When the sleeves 140 are placed in the couplings of the rotor mechanism 208, and a fluid is disposed in the fluid chamber 230, the sleeves 140 are in thermal communication with the fluid.

Preferably, the centrifuge 200 further includes a flow regulator 232 that is operable to receive fluid from a source 234 and control ingress, circulation, and/or evacuation of the fluid within the fluid chamber 230. For example, the flow regulator 232 may be electrically controllable, such that signals 236 thereto (and/or to associated ingress and egress ports) at least one of: (i) introduce the fluid into the fluid chamber 230; (ii) circulate the fluid within the fluid chamber 230, and (iii) evacuate the fluid from the fluid chamber 230. Such signals 236 may be produced via the microprocessor 220, memory 222, interfaces 224, and or input device 226 in response to appropriate programming and/or data as described above.

The centrifuge 200 may also include a heating mechanism 240 in thermal communication with the fluid chamber 230 (such as within the fluid chamber 230), which operates to regulate a temperature of the fluid and the material 10A within the sleeves 140. In this regard, the heating mechanism may include an electrically responsive heating element (such as a resistive heating element) that produces heat in response to an electrical current therethrough. In a preferred embodiment, the heating mechanism 240 produces heat in response to a signal or signals from the interface 224 on line 242. Such signals on line 242 may be produced via the microprocessor 220, memory 222, interfaces 224, and or input device 226 in response to appropriate programming and/or data as described above. In order to ensure suitable temperature regulation of the fluid (and thus the material 10A within the syringe 100 and sleeve 140), a temperature sensor 244 may be employed at an input or output port of the fluid chamber 230 to sense the fluid as it circulates. Alternatively, the temperature sensor 244 may be disposed within the chamber 230 itself. In any case, an electrical signal on line 246 produced by the temperature sensor 244 may be received by the micro-processor 220 via the interfaces 224. Under the control of suitable programming and/or data, the micro-processor 220 may utilize the information from the temperature sensor 244 to make adjustments in the signals on line 242 driving the heating mechanism 240.

When the sleeve 140 and the syringe 100 therein are within the fluid chamber 230 of the centrifuge 200, elevation of the temperature of the fluid within the chamber 230 elevates the temperature of the material 10A. Preferably the temperature of the material 10A is increased to a temperature sufficient to initiate or at least facilitate separation of the material 10A into strata. It has been found that elevation of the material 10A to about 37° C. for a suitable duration of time initiates or at least improves the stratification process. The heating time may be about 30 minutes, although as discussed below, other heating profiles may also be employed.

Although centrifugation will be discussed in more detail below, if the fluid within the fluid chamber 230 is a gas, such as air or some other gas, then the heating process may be conducted simultaneously with the centrifugation process. Indeed, as the sleeves 140 may rotate within the chamber 230, simultaneous application of heat may improve the stratification process as well as the separation process.

As discussed above, the mixture of materials 10A is preferably also subject to vibration whilst remaining within the syringe 100 to at least assist in the initiation or facilitation of separating the material 10A into strata. To this end, the centrifuge 200 also preferably includes a vibration mechanism 250 operatively coupled to the rotor mechanism 208 such that electrical drive signals to the vibration mechanism 250 cause vibration energy to be delivered to the sleeves 140, the syringe 100, and the material 10A therein.

With reference to FIG. 7, the vibration mechanism 250 may include a vibration motor 252 having a shaft 254 that rotates in response to the electrical drive signals on line 256. The shaft 254 is coupled to the intermediate platform 204 such that rotation of the shaft 254 imparts vibration movement thereto. The intermediate platform 204 may be coupled to the base 202 by way of one or more springs 258 (or other suitable coupling devices) such that the aforementioned vibration may be achieved whilst ensuring that there is suitable mechanical support for the structures coupled to the intermediate platform 204.

With reference to FIG. 8, one example is illustrated of a mechanism for converting the rotational movement of the shaft 254 into the vibration movement of the material 10A within the syringe 100 and sleeve 140. A cam 260 is coupled to an end of the shaft 254, where the cam 260 includes at least a semi-circular periphery (such as that of a circle) and a non-central axis of rotation. The intermediate platform 204 includes a cam follower 262, which may be an aperture, in engagement with the cam 260, such that rotation of the shaft 254 results in the vibration energy delivered to the intermediate platform 204. More particularly, with this example, the non-central axis of rotation of the cam 260 causes the cam follower 262 to follow an elliptical vibration path. The vibration of the intermediate platform 204 (and the path of the vibration) is coupled to the rotor mechanism 208, to the sleeves 140, to the syringe 100, and finally to the material 10A.

It is preferred that the signals driving the vibration motor 252 (e.g., on line 256) are provided by way of the microprocessor 220, memory 222, interfaces 224, and or input device 226 in response to appropriate programming and/or data as described above. In order to ensure suitable vibration regulation, a motion sensor 264 (such as an accelerometer) may be employed on the intermediate platform 204 (or other suitable surface) to sense the vibration characteristics being imparted by the motor 252. An electrical signal on line 266 produced by the motion sensor 264 may be received by the micro-processor 220 via the interfaces 224. Under the control of suitable programming and/or data, the micro-processor 220 may utilize the information from the motion sensor 264 to make adjustments in the signals on line 266 driving the motor 252.

If desired, the aforementioned heating process may be conducted simultaneously with the vibration process. This combined application of heat and vibration to the material 10A may, for example, be conducted for a time period (e.g., 30 minutes or so) prior to centrifugation. For example, if the fluid is a liquid, then the steps of heating and vibration may be conducted simultaneously, but the viscosity of the fluid might not allow for centrifugation. In such a case, the liquid is first drained (evacuated) from the fluid chamber 230 of the centrifuge 200. Thereafter, the centrifugation (possibly coupled with temperature regulation) may be carried out. Alternatively or additionally, the application of heat and vibration to the material 10A may be conducted simultaneously with the centrifugation process—especially if the fluid within the fluid chamber is a gas.

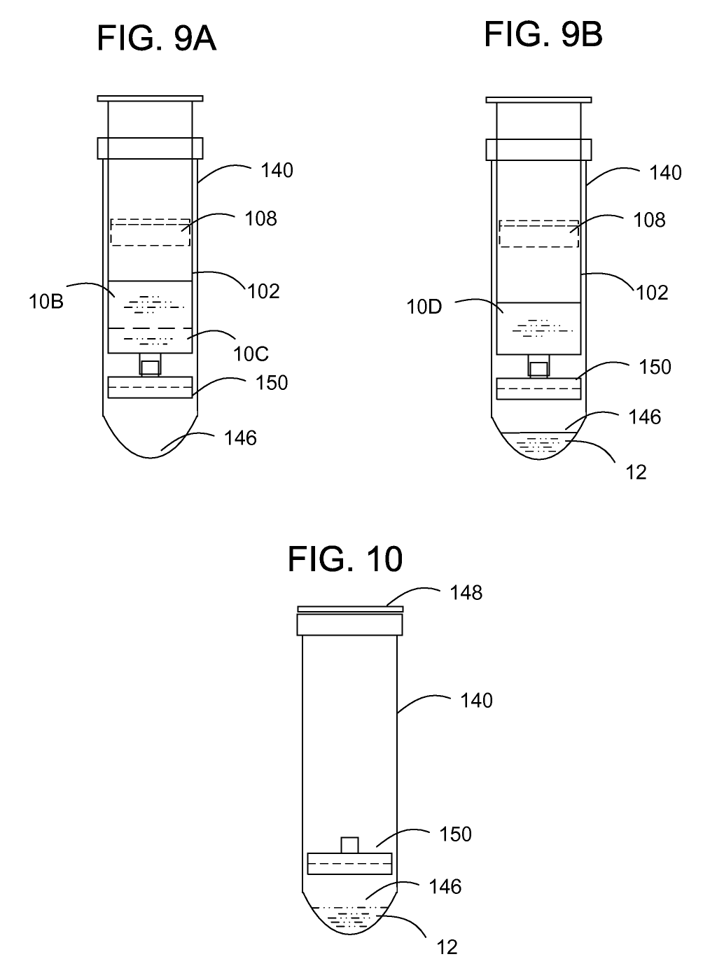

Irrespective of whether the heat and/or vibration are conducted before or during centrifugation, the material 10A is preferably subjected to centrifugation whilst still in the syringe 100 and the collection sleeve 140. As the material 10A experiences the centrifugal forces, it is driven toward and into the filter 150. With reference to FIGS. 9A and 9B, under proper regulation of the centrifugal speed of rotation and the duration of centrifugation, the mixture of materials 10A within the chamber 104 of the syringe 100 will stratify, with the oil and the fat generally being in one stratum 10B, and the denser materials, such as the tumescent fluid, ASCs, cell separation enzyme, and other substances being in another stratum 10C. More particularly, the material 10A may stratify into a top oil stratum, a middle fat stratum, and a bottom denser substance stratum (including the ASCs). This stratification takes place as a result of the differing densities of the components of the material 10A. For example, the oil may have the lowest density, followed by the densities of the fat. The fat may include less viable fat (for fat transplantation) which is of a lower density relative to the higher density of more viable fat tissue. The ASCs, tumescent fluid, collagenase, connective tissue, blood, and other non-fat substances have higher densities than the oil and fat.

The centrifugation may be conducted in accordance with a particular profile or profiles in order to achieve the aforementioned stratification. For example, centrifugation may be carried out for a particular period of time, such as for one of: (i) less than about 10 minutes; (ii) less than about 5 minutes, and (iii) about 2 minutes. It is believed, however, that centrifugation using a profile having a number of phases (one or more of which employing differing centrifugation durations and/or speeds/gravitational force) will yield satisfactory results. An example of such a profile is discussed later herein. G forces of 50 g's for removal of washes and 500-1000 g's for isolation of ASC's.

As mentioned above the centrifugation process causes the regenerative cells (ASCs) and some secondary materials (such as the tumescent fluid, collagenase, connective tissue, blood, and higher density materials) to be drawn toward and out of the distal end of the chamber 102 of the syringe 100 and into the filter 150. The mesh 154 prohibits materials with large size from passing through and out of the filter 150 into the reservoir 146. The ASCs, however, are of a size whereby they may pass into the reservoir 146. In addition, it is likely that at least some tumescent fluid, collagenase, and blood also passes through the filter 150, thus resulting in a material 12 within the reservoir 146 after centrifugation (FIG. 9B). The material 10D remaining in the syringe 100 includes viable fat that may be collected using other processes.

At least some of the tumescent fluid, the collagenase, the blood, and/or other materials are removed from the reservoir 146 of the collection sleeve 140 to obtain a concentration of the regenerative cells within the reservoir 146. This may be achieved using decanting processes, draining, filtering, etc. Thereafter, the collection sleeve 140 may be sealed via cap 148 and stored, preferably at a suitable temperature. Reconstitution of the ASCs may be achieved by adding a sterile fluid to the collection sleeve 140.

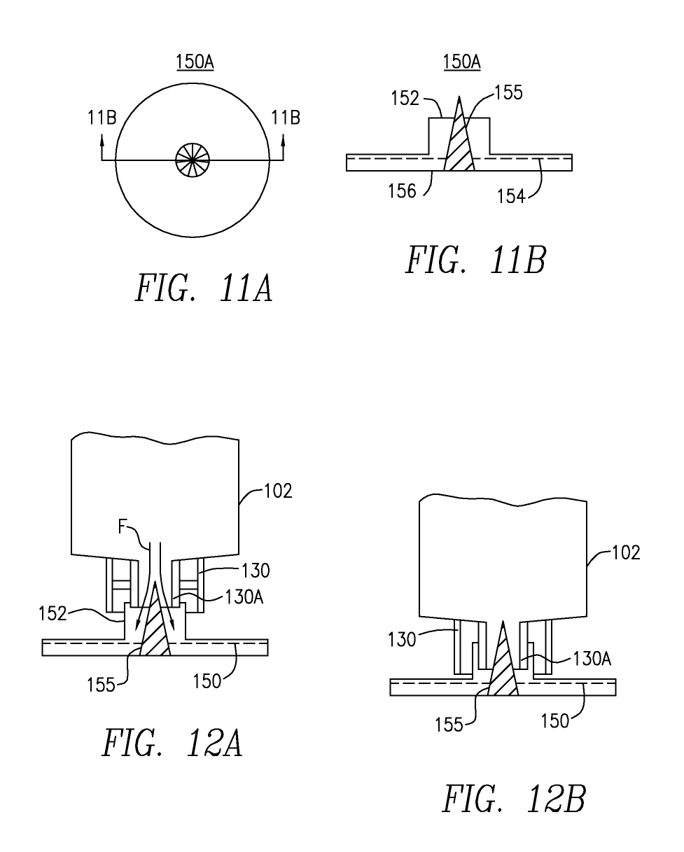

Reference is now made to FIGS. 11A-11B, and 12A-12B, which illustrate an alternative filter 150A, which includes features that permit opening and closing of the luer end of the syringe 100. Such features permit opening and closing the syringe 100 while same is disposed within the collection sleeve 140 and both are disposed within the centrifuge 200. This configuration is useful in carrying out certain steps in the centrifugation process. For example, centrifugation, vibration, and/or heating may be conducted for some period of time while the luer end of the syringe 100 is closed. Thus, one or more materials may be introduced and distributed within the chamber 104 (without fluids flowing out of the syringe 100) in order to facilitate the separation and processing of the ASCs. Such materials may include washing solutions, collagenase solution, injection media, etc.

FIG. 11A shows the filter 150A from a top view, and FIG. 11B shows the filter 150A in cross-section through line 11B-11B. The filter 150A is similar to the filter 150 discussed earlier herein. For example, the filter 150A includes an input end and an output end, the input end including a coupling 152 and corresponding aperture in fluid communication with an interior volume. The output end 156 includes one or more apertures 158 (as shown in FIG. 5B) also in fluid communication with the interior volume of the filter 150A. A mesh film 154 is disposed within the interior volume of the filter 150A, separating the input end from the output end thereof. The mesh 154 operates to pass material having a range of particle sizes and to block other material having another range of particle sizes. The filter 150A also includes a cone-shaped element 155 that is directed from the output end toward the input end thereof. The cone 155 is preferably long enough to extend toward, and in some configurations through, the coupling 152 of the filter 150A.

As illustrated in FIG. 12A-12B, when the body 102 of the syringe 100 is inserted into the open end 142 of the collection sleeve 140, the coupling 130 at the distal end of the syringe 100 mates with (e.g., via threads) the coupling 152 at the input end of the filter 150A. An aperture of an inner annular ring 130A of the coupling 130 is in fluid communication with the internal chamber 104 of the body 102 of the syringe 130. The cone 155 of the filter 155 extends into the aperture of the ring 130A, such that, at one or more first rotational orientations of the syringe 100 and the filter 150A, the cone 155 permits fluid from the chamber 104 to flow and pass into the inner volume of the filter 150A (FIG. 12A). In one or more second rotational orientations of the syringe 100 and the filter 150A (e.g., 180 degrees of rotation from the first orientation), however, the cone 155 engages against the ring 130A and prevents fluid from flowing out of the syringe 100 (FIG. 12B).

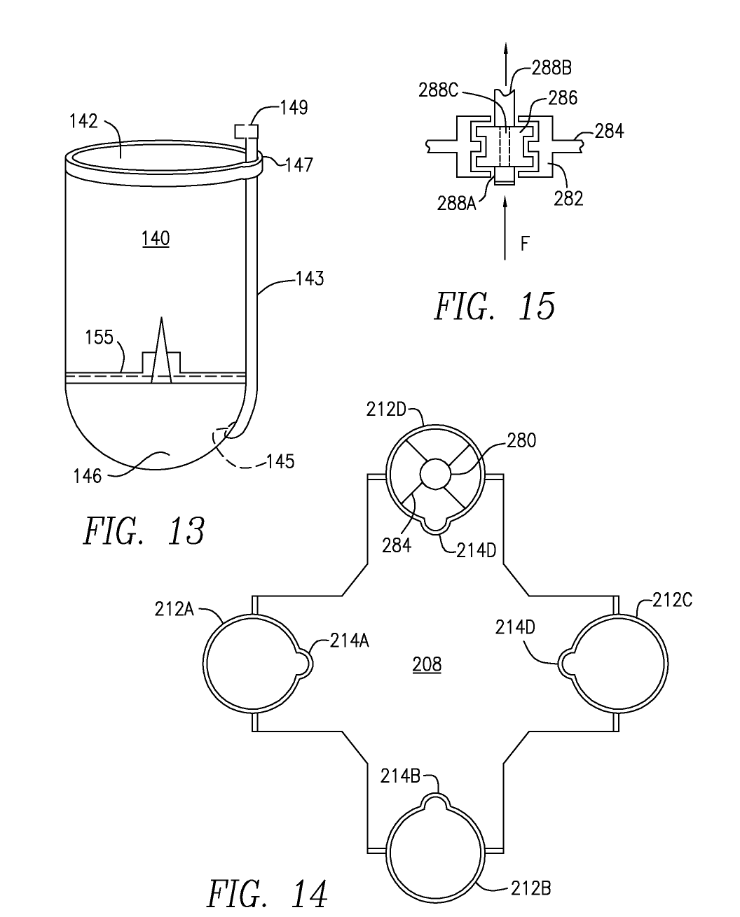

Reference is now made to FIGS. 13-15, which illustrate alternative configurations of a collection sleeve 140A, and swiveling rings 111 of the centrifuge 200. These configurations are useful in aspirating fluids from the reservoir 146 of the sleeve 140 during the centrifugation process.

As illustrated in FIG. 13, the collection sleeve 140A includes an aspiration port 143 having first and second opposite ends 145, 147. The first end 145 is in fluid communication with the reservoir 146 and the second end is disposed adjacent to the peripheral rim at the proximal end 142 of the sleeve 140. Preferably, the first end 145 is disposed some distance above a lowest end of the reservoir 146, such as about 5 mm up from the bottom thereof. The body of the port 143 (which is essentially a tube) extends from the first end 145 along the outside of the sleeve 140 to the second end 147. Alternative configurations may have the body of the port 143 extending along an inside of the sleeve 140, although such would also require that the filter 150, 150A, as well as the clearance between the syringe 100 and the inside of the sleeve 140, accommodate the geometry of the tube. The second end 147 of the port 143 may include a luer lock opening 149, which may be coupled to an aspiration port of a pump, etc. (not shown), such that refuse aspirated from the reservoir 146 may be removed and collected.

With reference to FIG. 14, the rotor mechanism 208 of the centrifuge 200 may include couplings 210 that employ special rings 212A, 212B, 212C, 212D, each such ring 212 including a respective receptacle 214A, 214B, 214C, 214D. The receptacles 214 are located about the rings 212 such they are centrally directed, i.e., they are closest to a center of rotation of the rotor mechanism 208. Thus, when the sleeves 140A are inserted into the rings 212, the respective ports 143 are received into the receptacles 214 and, thus, are also disposed closest to the center of rotation of the rotor mechanism 208. Thus, fluids will be driven up through the first end 145 of the tube of the port 143 and out the luer lock opening 149.

With reference to FIGS. 14-15, one or more of the rings 212 of the rotor mechanism 208 may include a universal ring 280. The universal ring 280 provides a way for fluid exiting the luer lock opening 149 of the port 143 to be carried to the aspiration pump and/or collection chamber while the rotor mechanism 208 is rotating. The universal ring 280 includes a housing 282 that is located at a central region of the ring 212 via arms 284, four such arms 284 being shown by way of example. A stator 286 is disposed within the housing 282 such that the housing 282 may rotate about the stator 286. The stator 286 includes an input end 288A, which is in fluid communication with the aspiration port 143, and an output end 288B, which is in fluid communication with the aspiration pump and/or collection chamber (e.g., via a tube, not shown). A central passage 288C extends through the stator 286 from the input end 288A to the output end 288B. Centrifugation drives fluid from the reservoir 146 of the sleeve 140A, through the port 143, and in the direction of the arrow F through the universal ring 280. Since the housing may rotate about the stator 286, such fluid flow may take place while the centrifuge 200 is operational and the rotor mechanism 208 is rotating.

The above embodiments of the present invention may be employed to carry out any number of profiles in order to achieve the aforementioned stratification, aspiration, and collection of ASCs. One such profile is discussed below.

The syringes 100 (having the material 10 therein) are placed within respective sleeves 140A (such that the coupling 130 is closed off by the cone 155 of the filter 150A), and the sleeves 140A are placed into the respective rings 212 of the rotor mechanism 208.

The syringes 100 are opened (by rotating the couplings 130 with respect to the filters 150A) and are subject to centrifugation at 50 g's of force for about two (2) minutes. During or after such centrifugation, all refuse is removed from the reservoirs 146 through the aspiration ports 143. This leaves adipocytes and adipose derived stem cells in the chambers 104 of the bodies 102 of the syringes 100.

The syringes 100 are then rotated with respect to the filters 150A to place them in the closed position. A washing solution (such as phosphate buffered saline with 1% antibiotic solution) is inserted into the chambers 104 of the syringes 100 (e.g., using the techniques described above with respect to FIGS. 2-3). Then the syringes 100 are shaken (as discussed with respect to FIGS. 6-8) for about two (2) minutes.

Next, the syringes 100 are rotated with respect to the filters 150A to place them in the open position. Then the syringes 100 are subject to centrifugation at about 50 g's for about two (2) minutes. During or after such centrifugation, all refuse is removed from the reservoirs 146 through the aspiration ports 143. It is noted that at this point, clean adipocytes and adipose derived stem cells are still in the respective chambers 104 of the syringes 100.

Next, the syringes 100 are rotated with respect to the filters 150A to place them in the closed position. A collagenase solution is inserted into the chambers 104 of the syringes 100 (e.g., using the techniques described above with respect to FIGS. 2-3). By way of example, the collagenase solution may include two separate packets that are combined prior to insertion into the syringes 100. The dry packet may include 0.01 mg of collagenase and 0.1 g of powdered bovine serum albumin, while the wet package may include 10 ml of phosphate buffered saline. Then the syringes 100 are shaken and heated (as discussed with respect to FIGS. 6-8) for about 30 minutes at a temperature of 37° C.

Next, the syringes 100 are rotated with respect to the filters 150A to place them in the open position. Then the syringes 100 are subject to centrifugation at about 300 g's for about five (5) minutes. During or after such centrifugation, all refuse is removed from the reservoirs 146 through the aspiration ports 143. It is noted that at this point, adipocytes remain in the chambers 104 of the syringes 100, and the adipose derived regenerative cells have moved through the filters 150A into the reservoirs 146 of the collection sleeves 140A along with the collagenase.

Next, the syringes 100 are removed from the collection sleeves 140A and the collagenase is removed from the reservoirs 146 via aspiration. The adipose derived regenerative cells will thus remain in the reservoirs 146 in pellet form, adherent to the bottoms of the respective reservoirs 146. A small amount of collagenase will also remain.

Next, a washing solution is added to the reservoirs 146, e.g., via the aspiration ports 143. Then the sleeves 140A are shaken for about two (2) minutes, followed by subjecting them to centrifugation at about 300 g's for about five (5) minutes. During or after such centrifugation, all refuse is removed from the reservoirs 146 through the aspiration ports 143. This will leave clean adipose derived regenerative cells in the reservoirs 146 in pellet form. As illustrated in FIG. 10, the adipose derived regenerative cell pellets may be stored for some period of time by placing caps 148 on the sleeves 140.

In order to reconstitute the regenerative cells from the pellets, one may add injection media (such as phosphate buffered saline through the aspiration ports 143 of the sleeves 140. Thereafter, the sleeves 140 are shaken for about two (2) minutes and spun at about 75 revolutions per minute (rpm) in order to reconstitute the cells. The regenerative cells may then be removed via the aspiration ports 143 with a sterile syringe for clinical use. Advantageously, within about 40-45 minutes, ASCs may be collected, processed and separated for clinical use without overly complex and costly machinery and with minimal risk of contamination (since the collected adipose material is processed within a closed system).

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A method, comprising:
collecting adipose tissue in a syringe, the syringe including a body having an internal chamber, a proximal end through which a plunger assembly slides into and out of the chamber, and a distal end through which the adipose tissue is drawn into the chamber;
inserting the body of the syringe into an open, proximal end of a collection sleeve such that the distal end of the syringe is in fluid communication with a reservoir at an opposing, closed end of the collection sleeve;
subjecting the collected adipose tissue to heat and vibration whilst remaining within the syringe in the collection sleeve to initiate separation of the adipose tissue into strata, where a concentration of the regenerative cells are in a first of the strata and a substantial concentration of fat is in a second of the strata;
subjecting the syringe and collection sleeve to centrifugation such that regenerative cells and some secondary materials in the first stratum are drawn toward and out of the distal end of the chamber of the syringe; and
filtering the first stratum such that the regenerative cells are permitted to pass to the reservoir of the collection sleeve in response to the centrifugation.

2. The method of claim 1, further comprising adding a cell separation enzyme to the collected adipose tissue within the syringe prior to heat and vibration.

3. The method of claim 2, further comprising:
separating a plunger shaft from a plunger head of the plunger assembly, thereby exposing a rear side of a resilient plunger within the chamber of the syringe;
inserting a needle or cannula through the open end of the syringe and through the resilient plunger; and
injecting the cell separation enzyme into the collected adipose tissue through the needle or cannula.

4. The method of claim 1, further comprising coupling the distal end of the syringe to a mating end of a filter disposed within the collection sleeve, the filter closing off the reservoir of the collection sleeve from the open, proximal end thereof, wherein the filter performs the filtering step by permitting the regenerative cells to pass through the mating end, through an output end thereof, and into the reservoir of the collection sleeve, but prohibits at least some of the secondary material from passing therethrough.

5. The method of claim 1, further comprising:
inserting the collection sleeve, the syringe, and the adipose tissue therein into a fluid chamber of a centrifuge;
elevating a temperature of fluid within the fluid chamber of the centrifuge to a predetermined temperature for a time sufficient to at least initiate separation of the adipose tissue into the strata.

6. The method of claim 5, wherein at least one of: the predetermined temperature is about 37° C.; and the time is about 30 minutes.

7. The method of claim 5, wherein the fluid is a gas and the step of heating and centrifugation are conducted simultaneously.

8. The method of claim 7, wherein the vibration is carried out simultaneously with the steps of heating and centrifugation.

9. The method of claim 5, wherein the fluid is a liquid and the step of centrifugation is conducted after the step of heating and after a step of draining the liquid from the fluid chamber of the centrifuge.

10. The method of claim 9, wherein the heat and vibration are conducted simultaneously.

10. The method of claim 9, wherein the step of centrifugation and heating are conducted simultaneously after the step of draining the liquid from the fluid chamber of the centrifuge.

11. The method of claim 1, wherein the step of centrifugation is conducted for one of: (i) less than about 10 minutes; (ii) less than about 5 minutes, and (iii) about 2 minutes.

12. The method of claim 1, further comprising removing at least one of tumescent fluid, collagenase, and blood from the collection sleeve to obtain a concentration of the regenerative cells within the reservoir.

13. A centrifuge, comprising:
a rotor having couplings for receiving sleeves to be subject to centrifugation; and
a vibration mechanism operatively coupled to the rotor such that electrical drive signals to the vibration mechanism cause the rotor to vibrate and deliver vibration energy to the sleeves.

14. The centrifuge of claim 13, wherein the vibration mechanism includes:
a vibration motor having a shaft that rotates in response to the electrical drive signals;
a cam coupled to the shaft of the vibration motor; and
a cam follower operatively coupled to the rotor and in engagement with the cam, such that rotation of the shaft results in the vibration energy delivered to the rotor.

15. The centrifuge of claim 14, wherein the cam and cam follower are sized and shaped such that the rotor vibrates in an elliptical pattern.

16. The centrifuge of claim 15, wherein the cam includes at least a semi-circular periphery and a non-central axis of rotation such that rotation of the shaft produces an elliptical vibration path in the cam follower.

17. The centrifuge of claim 13, further comprising a rotary motor operatively coupled to the rotor such that electrical drive signals to the rotary motor cause the rotor to spin and deliver centrifugal forces to the sleeves.

18. The centrifuge of claim 17, wherein the rotary motor and the vibration mechanism operate simultaneously such that samples within the sleeves are subject to both centrifugal forces and vibration forces.

19. The centrifuge of claim 13, further comprising a fluid chamber surrounding the rotor such that when the sleeves are placed in the rotor and a fluid is disposed in the fluid chamber, the sleeves are in thermal communication with the fluid.

20. The centrifuge of claim 19, further comprising a heating mechanism in thermal communication with the fluid chamber and operating to regulate a temperature of the fluid and samples within the sleeves.

21. The centrifuge of claim 19, further comprising ingress and egress ports and a flow regulator operating, in response to electrical signals, to at least one of: (i) introduce the fluid into the fluid chamber; (ii) circulate the fluid within the fluid chamber, and (iii) evacuate the fluid from the fluid chamber.
